(19) United States
(12) Patent Application Publication
(10) Pub. No.: US 2012/0295334 A1
(43) Pub. Date: Nov. 22, 2012

(54) METHOD FOR THE OXYGENATION OF FERMENTATIONS USING OXYGEN AND AIR OR OTHER OXYGEN REDUCING GASES

(75) Inventor: Sudhir R. Brahmbhatt, Glencoe, MO (US)

(73) Assignee: AIR LIQUIDE INDUSTRIAL U.S. LP, HOUSTON, TX (US)

(21) Appl. No.: 13/111,049

(22) Filed: May 19, 2011

(51) Int. Cl. C12N 1/20; C12M 1/34; C12M 1/36; C12M 1/00

(52) U.S. Cl. 435/252.5; 435/289.1; 435/287.5; 435/286.6

(57) ABSTRACT

An improved fermentation device and method uses a combination of substantially pure Oxygen as the Oxygenation gas and a separate air (or other Oxygen reducing gas) purge through the headspace of a fermentation device. The air injection and air purge vent line are configured to maintain a backpressure within the headspace. The air purge permits improved oxygen utilization and increases safety through dilution and removal of ammonia gas emitted during fermentation.

### TECHNICAL FIELD

The technical field is control of Oxygenation for aerobic fermentations.

### BACKGROUND ART

The prior art is represented by US 2007/0087402 A1“Oxygen-Assisted Fermentation Process.” This application describes the use of substantially pure Oxygen as the Oxygen gas instead of air or an air/Oxygen blend. The benefits of this system include increased Oxygen transfer efficiency and significantly higher dissolved Oxygen levels. These benefits directly translate into enhanced fermentation productivity and higher yields.

US 2007/0087402 A1 is hereby incorporated by reference in its entirety. US 2007/0087402 A1 represents as safe and effective improvement over air based Oxygenation systems. However, for some fermentations, problems exist with this technology that partially diminish the benefits.

In general, Oxygen is relatively expensive compared to air. Thus it would be valuable to reduce Oxygen utilization without sacrificing Oxygenation rates or dissolved Oxygen levels.

Another aspect of the prior art that represents a challenge is ammonia which is produced from Nitrogen based feed (e.g. Ammonium salts, nitrate salts, urea, amino acids, NH₄ etc.) normally used in fermentation. The improved fermentation capacity resulting from using substantially pure Oxygen can lead to more ammonia production and may result in high ammonia levels in the head space of a fermentation system. This ammonia in the presence of possible high Oxygen levels in the head space does present a safety issue when substantially pure Oxygen is used in media. This is a general problem with typical aerobic fermentation and in particular with Bacillus based fermentation.

Safety issues regarding ammonia include human exposure. Human exposure dangers are well known. Hazardous Chemicals desk reference, by N. Irving Sax/Richard J. Lewis, Sr., pg. 159. Because of the adverse effects of ammonia exposure, various standard for worker exposure have been defined including:

TWA (Time Weighted Average) 25 ppm, concentration in air to which workers can be exposed for a normal 8 hr work day for 40 hrs week without any ill effect.

STEL (Short Tem exposure limit) 35 ppm Time weighted average concentration in air which should not be exceeded.

OSHA PEL (OSHA Permissible Exposure Limit) 50 ppm Time Weighted Average for which workers can be exposed for a normal 8 hrs day.

A separate hazard issue with ammonia is explosion in the presence of Oxygen. Oxygen and ammonia levels can reach dangerous concentrations in the gasses coming out a fermentation media Oxygenated with substantially pure Oxygen. Oxygen enriched ammonia is in fact a renewable alternative fuel to petrochemical fuels (promoted for example by the NH3 Fuel Association). Most fermentation operations however will not be equipped to safely handle explosive ammonia/Oxygen blends produced as a consequence of using substantially pure Oxygen for Oxygenation of the medium.

These and other problems are addressed by the improvements described herein.

### BRIEF SUMMARY OF THE INVENTION

The following paragraphs define aspects and embodiments within the scope of the invention. As used in the Brief Summary of the Invention, “further consisting of” refers to a device or process consisting of the referenced paragraph's or paragraphs' element(s) and those element(s) following the phrase in the same paragraph.

An improved fermentation apparatus comprising, consisting essentially of, or consisting of:
a. a fermentation tank 10 containing within the tank 10 a fermentation medium 150 and having a headspace 110 within the tank 10 above the fermentation medium 150,
b. a substantially pure Oxygen supply system 40, 50, 60 in fluid communication with an interior cavity of the fermentation tank 10 through a gas diffuser 30,
   wherein the fluid communication of the substantially pure Oxygen supply system 30, 40 is configured to inject substantially pure Oxygen into the fermentation medium 150 within the fermentation tank 10,
c. an air compression device 100, in fluid communication 160 with the headspace 110 of the fermentation tank 10,
wherein the fluid communication 160 of the air compression device 100 is separate from the fluid communication of the substantially pure Oxygen supply system 30, 40, and
wherein the fluid communication 160 of the air compression device 100 is configured to inject a pressurized air directly into the headspace 110 of the fermentation tank 10.

The fermentation apparatus of paragraph [0016] further comprising, consisting essentially of, or consisting of a dissolved Oxygen probe 90 configured to be capable of measuring a dissolved Oxygen level of the fermentation medium 150 contained within the fermentation tank 10.

The fermentation apparatus of paragraph [0016] or [0017], wherein a flow rate of Oxygen 50, 60 from the substantially pure Oxygen supply system 30, 40, 60 into the fermentation tank 10 is controlled by a specifically programmed computer adapted to adjust the flow rate of Oxygen 50, 60 in response to a dissolved Oxygen measurement 90 of the fermentation medium 150.

The fermentation apparatus of paragraph [0016], [0017] or [0018], wherein the substantially pure Oxygen supply system 30, 40, 50, 60 is the only source of Oxygen configured to be capable of injecting an Oxygen containing gas into the fermentation medium 150 within the fermentation tank 10.

The fermentation apparatus of paragraph [0016]-[0018] or further comprising, consisting essentially of, or consisting of a vent line 130 in fluid communication with the headspace 110, a backpressure regulator 120 operably connected to the vent line 130, and an exhaust gas analysis device 140 configured to take gas samples from the vent line 130 and/or the backpressure regulator 120 and capable of measuring one or more of a Carbon Dioxide level, an Oxygen level, or an ammonia level in the gas samples.

The fermentation apparatus paragraph [0020] further comprising, consisting essentially of, or consisting of a specifically programmed computer
a. configured to be capable of receiving the Oxygen level and/or the ammonia level from the exhaust gas analysis device 140, and
b. configured to control
   i. a rate of air injection 100, 160 into the headspace 110 in response to Oxygen level and/or the ammonia level 140, and/or
   ii. a rate of air flow through the backpressure regulator 120 in response to the Oxygen level and/or the ammonia level 140.

The fermentation apparatus of paragraph [0021], wherein the specifically programmed computer is configured to control b)i) or ii) based on the Oxygen level and/or the ammonia level required to avoid a combustible mixture.

The fermentation apparatus of paragraph [0022], wherein the combustible mixture is defined as having one or more of a) an ignition temperature of >400 degrees C., b) an ammonia concentration of 15% or higher, and c) an Oxygen concentration of more than 21%.

The fermentation apparatus of paragraphs [0021], [0022] or [0023], wherein the specifically programmed computer is configured to control b)i) or ii) based on the Oxygen level and/or the ammonia level required to avoid a hazardous human exposure level of ammonia.

The fermentation apparatus of paragraph [0024], wherein the hazardous human exposure level is defined as 25 ppm ammonia or higher.

The fermentation apparatus of paragraph [0016]-[0024] or [0025], further comprising, consisting essentially of, or consisting of a vent line 130 terminating at a point in fluid communication with the outdoors atmosphere.

The fermentation apparatus of paragraph [0016]-[0025] or [0026], further comprising, consisting essentially of, or consisting of a second purge gas source in fluid communication with the the air compression device 100 and optionally a mixing device positioned between the headspace 110, the air compression device 100 and second purge gas sources such that, during operation of the apparatus, the mixing device is capable of mixing the air 100 and second purge gas prior to the air 160 being injected into the headspace 110.

An improved fermentation medium 150 Oxygenation process further comprising, consisting essentially of, or consisting of the steps of
a. injecting substantially pure Oxygen 30, 40, 50, 60 into the fermentation medium 150 contained within a fermentation tank 10,

b. injecting a gas 100 having an Oxygen content of 21% or less into a headspace 110 within the fermentation tank 10,

c. releasing gases 120, 130 from the headspace 110, and

d. maintaining a gas pressure in the headspace 110 at greater than atmospheric pressure.

The process of paragraph [0028] further comprising, consisting essentially of, or consisting of the steps of controlling the rates of injecting gas 100 and/or releasing gases 120, 130 to maintain an ammonia level and/or an Oxygen level in the released gases 120, 130 at or below a desired level.

The process of paragraph [0028] or [0029] wherein substantially pure Oxygen 30, 40, 50, 60 is the only Oxygen containing gas injected into the fermentation medium 150.

The process of paragraph [0028], [0029] or [0030] further comprising, consisting essentially of, or consisting of the step of controlling a rate of injecting substantially pure Oxygen 30, 40, 50, 60 to maintain a dissolved Oxygen level 90 in the fermentation medium 150 at or above a desired level.

The process of paragraph [0029], [0030], or [0031], wherein the desired ammonia and/or Oxygen level is a level that corresponds to a noncombustible gas mixture.

The process of paragraph [0032], wherein the noncombustible mixture is defined as having one or more of a) an ignition temperature of >400 degrees C., b) an ammonia concentration of 15% or less, and c) an Oxygen concentration of 21% or less.

The process of paragraph [0029]-[0032], or [0033], wherein the desired ammonia level is below a human exposure hazard level.

The process of paragraph [0034], wherein the hazardous human exposure level is defined as 25 ppm ammonia or higher.

The process of paragraph [0028]-[0034], or [0035], wherein step c) comprises, consists essentially of, or consists of the step of venting the gases 120, 130 to an exterior atmosphere.

The process of paragraph [0028]-[0035], or [0036], wherein the injected gas 100, 160 comprises, consists essentially of, or consists of air.

The process of paragraph [0028]-[0036], or [0037], further comprising combining a second gas with the air 100, 160 prior to step b) to thereby reduce an Oxygen content of the combined gas to below the Oxygen content of the air 100.

The process of paragraph [0038], wherein the second gas comprises, consists essentially of, or consists of a Nitrogen gas having less than a 21% Oxygen content.

The fermentation apparatus of paragraph [0016]-[0026] or [0027], wherein the air compressor 100 is replaced with a pressurizing source of substantially pure Nitrogen having a Nitrogen content >80%, preferably at least 90%, such as 90%, 93%, 95% or 99% Nitrogen.

An improved fermentation apparatus comprising, consisting essentially of, or consisting of:
a. a fermentation tank 10 containing within the tank 10 a fermentation medium 150 and having a headspace 110 within the tank 10,
b. means for supplying substantially pure Oxygen 30, 40, 50, 60 into the fermentation medium 150,
c. means for delivering a pressurized air 100, 160 directly into the headspace 110,
wherein means for delivering a pressurized air 100, 160 is separate from means for supplying substantially pure Oxygen 30, 40, 50, 60.

The fermentation apparatus of paragraph [0041] further comprising, consisting essentially of, or consisting of a means for measuring dissolved Oxygen 90 in the fermentation medium 150 contained within the fermentation tank 10.

The fermentation apparatus of paragraph [0041] or [0042], further comprising, consisting essentially of, or consisting of a means for adjusting a flow rate of Oxygen 50, 60 in response to a dissolved Oxygen 90 of the fermentation medium 150.

The fermentation apparatus of paragraph [0041], [0042] or [0043], wherein the means for supplying substantially pure Oxygen 30, 40, 50, 60 is the only means of the apparatus for injecting an Oxygen containing gas into the fermentation medium 150 within the fermentation tank 10.

The fermentation apparatus of paragraph [0041]-[0043] or [0044] further comprising, consisting essentially of, or consisting of a means for venting 120, 130 gas from the headspace 110, means for maintaining a backpressure 120 in the headspace, and an exhaust gas analysis means 140 for analyzing one or more of a Carbon Dioxide level, an Oxygen level, or an ammonia level in the gas from the headspace 110.

The fermentation apparatus paragraph [0041]-[0044] or [0045] further comprising, consisting essentially of, or consisting of
a. means for receiving the Oxygen level and/or the ammonia level from the exhaust gas analysis means 140, and
b. means for controlling
   i. a rate of air injection 100 into the headspace 110 in response to the Oxygen level and/or the ammonia level, and/or
   ii. a rate of air flow through the backpressure regulator means 120 in response to the Oxygen level and/or the ammonia level.

The fermentation apparatus of paragraph [0046], wherein the means for controlling b)i) or ii) controls for an Oxygen level and/or an ammonia level required to avoid a combustible mixture and/or required to avoid a hazardous human exposure level of ammonia.

The fermentation apparatus of paragraph [0047], wherein the combustible mixture is defined as having one or more of a) an ignition temperature of >400 degrees C., b) an ammonia concentration of 15% or higher, and c) an Oxygen concentration of more than 21%.

The fermentation apparatus of paragraph [0047], wherein the hazardous human exposure level is defined as 25 ppm ammonia or higher.

The fermentation apparatus of paragraph [0041]-[0048] or [0049], further comprising, consisting essentially of, or consisting of a venting means 130 for venting to the outdoors atmosphere.

The fermentation apparatus of paragraph [0041]-[0049] or [0050], further comprising, consisting essentially of, or consisting of a second gas means for delivering a second gas to combine with the air from the means for delivering a pressurized air 100, 160 and optionally a mixing means for mixing the air 100 and the gas from second gas source prior to injection into headspace 110.

### DESCRIPTION OF THE DRAWING

FIG. 1 illustrates an embodiment or the apparatus suitable for use in some embodiments of the method.

### DISCLOSURE OF INVENTION

The improved fermentation device and method implement a combination of substantially pure Oxygen as the Oxygenation gas and a separate air purge injected only into the headspace of the fermentation device. The air purge rate is adjusted to maintain a desired backpressure within the headspace. As a consequence, dissolved Oxygen levels per unit of Oxygen input are improved relative to the prior design without the air purge. This surprisingly results in a more efficient and economical use of Oxygen by maintaining pressurized air in the headspace.

The air or inert gas such as nitrogen gas purge of the headspace has the added benefit of diluting out ammonia emanating from the microbial use of a nitrogen based feed in a fermentation. The dilution of ammonia by the air purge ensures that ammonia levels in the effluent gas are maintained within safe limits. For Bacillus fermentation using Oxygen, the management of ammonia levels in the headspace and effluent gas is particularly important.

### BRIEF DESCRIPTION OF DRAWINGS

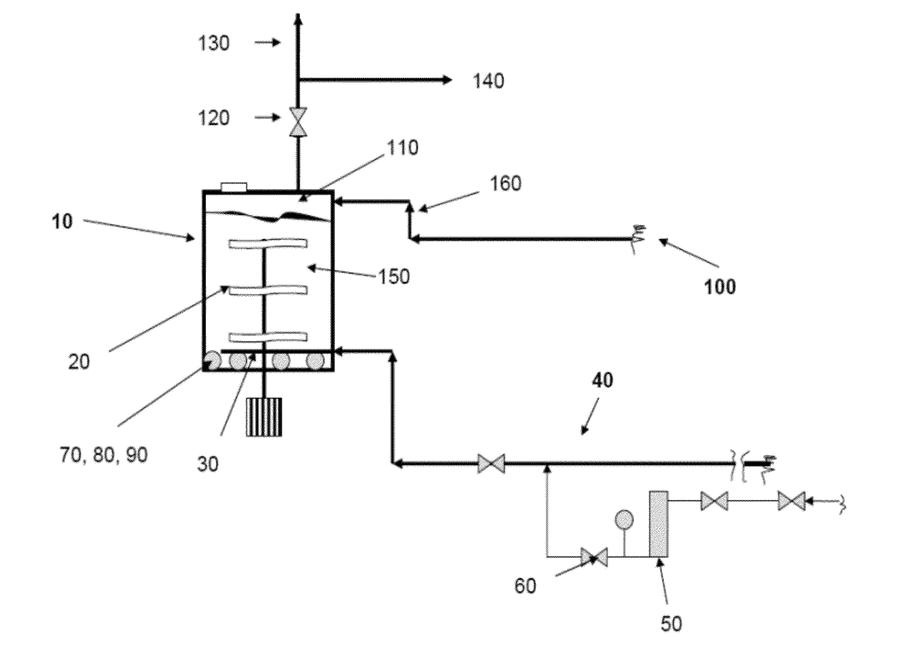

FIG. 1 shows an exemplary mechanical agitation fermenter 10, 20 with an air compressor 100 purging air under pressure into the headspace 110 above the fermentation medium 150.

### MODE(S) FOR CARRYING OUT THE INVENTION

With reference to FIG. 1, a standard mechanical agitation, fermentation tank 10 is shown having an agitation impeller 20.

“Substantially pure Oxygen” has an Oxygen purity of at least 80% or more, preferably 90% or more Oxygen, such as commercially available 93% Oxygen or 99.5% Oxygen. Substantially pure Oxygen is injected into the fermentation medium 150 through gas diffusers 30. The diffusers 30 may be porous or perforated pipes. Preferably, the diffusers 30 are positioned to emit Oxygen bubbles beneath the impellor 20 to maximize Oxygen bubble sheering and mixing with the fermentation medium 150. The target dissolved Oxygen levels for a particular device and fermentation media are generally determined empirically. The flow of Oxygen is controlled by the Oxygen supply system 40 which has an Oxygen flow meter 50 and solenoid valve 60. Within the tank 10, there are a series of probes for pH 70, temperature 80, and Oxygen 90. Oxygen probe 90 measures dissolved Oxygen levels in the fermentation medium 150. The Oxygen supply system 40 may be adjusted manually or through action of a specifically programmed computer. Adjustment of solenoid valve 60 for example may be automated in response to dissolved Oxygen measurements from probe 90 to increase or decrease Oxygen flow rates as measured by Oxygen flow meter 50.

The air compression device 100 may be any air compression device as is known in the art for use in delivering air to a fermentation system. Such compressors are generally reciprocating, rotary or centrifugal compressors with a particulate-filter and a sterilizing filter. The main concern with selection of an air compressor 100 is that the air compressor 100 have a sufficient discharge pressure through air conduit 160 to both purge the headspace 110 of the fermentation tank 10 and overcome the back pressure in the headspace 110 caused by the back pressure regulator 120.

Fermentation tank 10 has a vent line 130 in fluid communication with headspace 110. Back pressure regulator 120 controls the rate of air flow through the vent line 130 to maintain a desired backpressure within the headspace 110 in conjunction with the pressurizing input of air from the air compression device 100.

The target headspace 110 pressure for a particular device and fermentation media are generally determined empirically. The degree of pressure will be confined by the available input pressure 100 and the maximum pressure safely contained by a specific fermentation tank 10 and/or the back pressure regulator 120. In generally, the headspace pressure will be greater than one standard atmosphere and/or greater than the actual ambient atmospheric pressure. Within this range, the optimum pressure will generally correspond to the least pressure required to attain the maximum increase in Oxygen transfer to the fermentation medium 150 and/or the maximum increase in dissolved Oxygen 90, these levels being measured relative to fermentation without purge air 100, 160 and headspace backpressure 120.

The level of Oxygen, ammonia and the reaction initiation temperature are some variables affecting whether an ammonia/Oxygen blend will explode. The initiating thermal energy is generally high enough that gases emitted by a fermentation culture would normally be exposed to such temperatures only by accident. The variables therefore within operator control are the ammonia levels and Oxygen levels 140 in the gases emanating from the fermentation medium 150.

For a fermentation emitting ammonia, the flow rate of air 100, 160 through headspace 110 should be controlled to maintain a safe level of ammonia and/or Oxygen in the effluent gas 140. Ammonia and/or Oxygen may be monitored by an exhaust gas analysis device 140 in fluid communication with the vent line 130.

Safe ammonia levels may be defined by human exposure limit requirements such as a maximum of 25 ppm, 35 ppm, or 50 ppm ammonia.

Safe levels may also be defined as mixtures of Oxygen and ammonia that are non-combustible or at least requiring an ignition temperature of >400 degrees C. such as >585, >630 degrees C., >700 degrees C., >1000 degrees C., or >1300 degrees C.

As discussed above, ammonia combustion in Oxygen is affected by the concentrations of both in a gaseous mixture. In an ideally balanced fermentation using substantially pure oxygen, the Oxygen input 30, 40 will maintain the desired dissolved Oxygen levels 90 without significant amounts of Oxygen gas being emitted into headspace 110. In operation, there are many times when Oxygen input 30, 40 will at least transiently exceed the necessary input levels. For example, at the start of fermentation, Oxygen input levels may be deliberately set higher than necessary and then calibrated back to the optimum input level. Another point in fermentation will be the shift from exponential growth to stationary growth. Oxygen input 30, 40 may transiently exceed the requirement for the target dissolved oxygen level 90 due to decreases in Oxygen consumption by the fermentation microorganisms. During such adjustments periods, Oxygen concentrations 140 in the headspace 110 may be very high such as 25 or 30% of the headspace 110 gases. At the same time, ammonia levels can be emitted at high levels as well. Ammonia for example is combustible when ammonia is mixed 15%-28% (112,280 ppm-209,600 ppm) by volume with air. Such levels of ammonia are possible in a closed fermentation system due to the dramatic increase in production capacity resulting from use of substantially pure Oxygen. A transient spike in Oxygen passing through the fermentation medium 150 into the headspace 110 could easily produce a combustible gas mixture. Thus, air purging 100, 120, 130, 160 of headspace 110 may be calibrated to maintain the ammonia level 140 below 15%. Alternatively, air may be used to reduce Oxygen levels 140 in the headspace 110 to approximately 21% or less. During normal operations with low Oxygen emissions form the fermentation medium, the normal carbon dioxide emission from fermentation in combination with air purging 100, 120, 130, 160 may maintain the Oxygen level 140 in the headspace below 21% such as from 20.9% to 19%. Reduction of Oxygen and ammonia levels 140 are complementary and thus can be simultaneously affected by the air purge 100, 120, 130, 160 of the headspace 110. With the air purge 100, 120, 130, 160 of the invention, ammonia levels 140 may be kept far below combustible levels such as at 225 ppm or less and Oxygen levels 140 may in many instances be maintained below 21% due to dilution by concurrent carbon dioxide emissions from the fermentation medium 150.

In some circumstances, use of less than the maximum dissolved Oxygen levels 90 possible may be required to ensure sufficient dilution of ammonia 140. However, increased pressure in the headspace 110 actually increases the dissolved Oxygen 90 saturation point of the fermentation media 150. For example, at 1 atm pressure, the dissolved Oxygen maximum is 60 ppm at 6° C. while it is 72.5 ppm at 2 atm pressure at same temperature. Thus an air pressurized headspace 110 can advantageously increase the efficiency of use of substantial Oxygen while simultaneously mitigating the hazards due to the resultant increase in ammonia production.

As an additional safety feature, the vent line 130 may terminate outside of a the fermentation facility and be equipped with a fan or other mixing device to use the external atmosphere to further dilute the ammonia and/or Oxygen levels 140 for example to further reduce ammonia levels 140 to below safe human exposure limits.

While the foregoing has been described in the context of a mechanical agitation fermenter 10, 20, the device may be a non-agitation air-lift fermenter or any other fermenter configuration known in the art. The foregoing describes the use of air 100 as the purge gas; however other purge gases having the same amount or less Oxygen than air 100 (i.e. less than 21% Oxygen) may be blended with or fully substituted for air 100. For example substantially pure Nitrogen gas may be blended with air 100 to further reduce the Oxygen concentration prior to injection into headspace 110. This may be readily accomplished by connecting a Nitrogen source to the air source 100 and optionally feeding the mixture of air and Nitrogen through a gas mixing device. Blending or substituting air 100 with a lower Oxygen content gas will generally not be required but may be needed in specific circumstances to reduce or eliminate a risk of combustible gases mixtures forming in headspace 110.

### EXAMPLE

An aerobic fermentation process to produce Bacillus bacteria biomass was carried out with air as Oxygen source in the media. As the Bacillus bacteria population went up, the demand for oxygen in the media went up and the Dissolved Oxygen (DO) started dropping. The oxygen transfer rate in the media depends on either the mass transfer coefficient and/or oxygen concentration gradient. But, the concentration gradient for oxygen is set at a given temperature and pressure and is low when air is used. Hence, the only way to increase the oxygen transfer rate and attempt to maintain dissolved Oxygen levels is to increase the mass transfer coefficient and this was accomplished by high agitation rates and/or high air flow rates. For comparison, substantially pure Oxygen was used in place of air and the headspace was purged with air to build up slight pressure and dilute ammonia according to the invention. Results:
Foaming was essentially eliminated.
DO was easily maintained and the cell density increased significantly in a short time enabling the user to achieve results in a significantly short time.
The DO and Carbon Dioxide levels were monitored in the vent line to study the cell growth.

The use of pressurized air 100 in the headspace 110 allowed optimum Oxygen input to maximize Oxygen transfer rates and Oxygen utilization rates. This further enhanced the safety of the operation by diluting the ammonia that may be present in the head space 110 of fermenter.

### INDUSTRIAL APPLICABILITY

The invention has industrial applicability to the control of Oxygenation for aerobic fermentations to produce microbial biomass and/or fermentation products.

1. An improved fermentation apparatus comprising
a) a fermentation tank containing within the tank a fermentation medium and having a headspace within the tank,
b) a substantially pure Oxygen supply system in fluid communication with an interior cavity of the fermentation tank through a gas diffuser,
i) wherein the fluid communication of the substantially pure Oxygen supply system is configured to inject substantially pure Oxygen into the fermentation medium within the fermentation tank,
c) an air compression device, in fluid communication with the headspace of the fermentation tank,
i) wherein the fluid communication of the air compression device is separate from the fluid communication of the substantially pure Oxygen supply system and
ii) wherein the fluid communication of the air compression device is configured to inject a pressurized air directly into the headspace of the fermentation tank.

2. The fermentation apparatus of claim 1 further comprising a dissolved Oxygen probe configured to be capable of measuring a dissolved Oxygen level of the fermentation medium contained within the fermentation tank.

3. The fermentation apparatus of claim 2, wherein a flow rate of Oxygen from the substantially pure Oxygen supply system into the fermentation tank is controlled by a specifically programmed computer adapted to adjust the flow rate of Oxygen in response to a dissolved Oxygen measurement of the fermentation medium.

4. The fermentation apparatus of claim 1 wherein the substantially pure Oxygen supply system is the only source of Oxygen configured to be capable of injecting an Oxygen containing gas into the fermentation medium within the fermentation tank.

5. The fermentation apparatus of claim 1 further comprising a vent line in fluid communication with the headspace, a backpressure regulator operably connected to the vent line, and an exhaust gas analysis device configured to take gas samples from the vent line and capable of measuring one or more of a Carbon Dioxide level, an Oxygen level, or an ammonia level in the gas sample.

6. The fermentation apparatus of claim 5 further comprising a specifically programmed computer
a) configured to be capable of receiving the Oxygen level and/or the ammonia level from the exhaust gas analysis device, and
b) configured to control
i) a rate of air injection into the headspace in response to the Oxygen level and/or the ammonia level, and/or
ii) a rate of air flow through the backpressure regulator in response to the Oxygen level and/or the ammonia level.

7. The fermentation apparatus of claim 6, wherein the specifically programmed computer is configured to control b)i) or ii) based on the Oxygen level and/or the ammonia level required to avoid a combustible mixture.

8. The fermentation apparatus of claim 7 wherein the combustible mixture is defined as having one or more of a) an ignition temperature of >400 degrees C., b) an ammonia concentration of 15% or higher, and c) an Oxygen concentration of 21% or higher.

9. The fermentation apparatus of claim 6, wherein the specifically programmed computer is configured to control b)i) or ii) based on the Oxygen level and/or the ammonia level required to avoid a hazardous human exposure level of ammonia.

10. The fermentation apparatus of claim 9, wherein the hazardous human exposure level is defined as 25 ppm ammonia or higher.

11. The fermentation apparatus of claim 5 further comprising the vent line terminating at a point in fluid communication with an outdoors atmosphere.

12. The fermentation apparatus of claim 1 further comprising a second purge gas source in fluid communication with the air source and optionally a mixing device positioned between the headspace and the air and second purge gas sources such that, during operation of the apparatus, the mixing device is capable of mixing the air and the second purge gas prior to the mixture being injected into the headspace.

13. An improved fermentation medium Oxygenation process comprising the steps of
a) injecting substantially pure Oxygen into the medium contained within a fermentation tank,
b) injecting a gas having an Oxygen content of 21% or less into a headspace within the fermentation tank,
c) releasing gases from the headspace, and
d) maintaining a gas pressure in the headspace at greater than atmospheric pressure.

14. The process of claim 13 further comprising the steps of controlling the rates of injecting gas and/or releasing gases to maintain an ammonia level and/or an Oxygen level in the released gases at or below a desired level.

15. The process of claim 13 wherein substantially pure Oxygen is the only Oxygen containing gas injected into the fermentation medium.

16. The process of claim 13 further comprising the step of controlling a rate of injecting substantially pure Oxygen to maintain a dissolved Oxygen level in the fermentation medium at or above a desired level.

17. The process of claim 13, wherein the desired ammonia and/or Oxygen level is a level that corresponds to a noncombustible mixture defined as having one or more of a) an ignition temperature of >400 degrees C., b) an ammonia concentration of 15% or less, and c) an Oxygen concentration of 21% or less.

18. The process of claim 13, wherein step c) comprises venting the gases to an exterior atmosphere.

19. The process of claim 13 wherein the injected gas comprises air.

20. The process of claim 19 further comprising combining a second gas with the air prior to step b) to thereby reduce an Oxygen content of the combined gas to below the Oxygen content of the air.
